(19) United States
(12) Patent Application Publication
(10) Pub. No.: US 2023/0147052 A1
(43) Pub. Date: May 11, 2023

(54) METHODS AND COMPOSITIONS FOR INDUCING AUTOPHAGY

(71) Applicant: SELECTA BIOSCIENCES, INC., Watertown, MA (US)

(72) Inventors: Petr Ilyinskii, Combridge, MA (US); Takashi Kei Kishimoto, Lexington, MA (US)

(21) Appl. No.: 17/918,706

(22) PCT Filed: Apr. 14, 2020
(86) PCT No.: PCT/US2020/028132

§ 371 Date: Oct. 13, 2022

(51) Int. Cl. A61K 31/436; A61P 37/06

(52) U.S. Cl. CPC A61K 31/436; A61P 37/06

(57) ABSTRACT

Provided herein are methods and compositions related to compositions comprising synthetic nanocarriers comprising an immunosuppressant. Also provided herein are methods and compositions for inducing or increasing autophagy.

### FIELD OF THE INVENTION

Provided herein are methods and compositions related to synthetic nanocarriers comprising an immunosuppressant for inducing autophagy. The compositions and methods may be used to treat or prevent autophagy-associated diseases or disorders, for example.

### SUMMARY OF THE INVENTION

In one aspect, provided herein are methods for inducing or increasing autophagy in a subject comprising administering a composition comprising synthetic nanocarriers comprising an immunosuppressant to the subject. In one embodiment, the subject is one in need of the induction or increase in autophagy.

In one aspect, provided herein are methods for treating or preventing an autophagy-associated disease or disorder in a subject comprising administering a composition comprising synthetic nanocarriers comprising an immunosuppressant to the subject, wherein the subject has or is at risk of developing an autophagy-associated disease or disorder.

In one embodiment of any one of the methods provided, the administration of the synthetic nanocarriers comprising the immunosuppressant induces autophagy (e.g., modulates the levels of ATG7, LC3II, and/or p62).

In one embodiment of any one of the methods provided, administration of the synthetic nanocarriers comprising the immunosuppressant increases autophagy in the liver.

In one embodiment of any one of the methods provided, the synthetic nanocarriers comprising the immunosuppressant are not administered concomitantly with a therapeutic macromolecule or are administered concomitantly with a combination of a therapeutic macromolecule and a separate administration (e.g., not in the same administered composition and/or administered separately for a different purpose such as not for inducing or increasing autophagy) of synthetic nanocarriers comprising an immunosuppressant. In one embodiment of any one of the methods provided, the synthetic nanocarriers comprising the immunosuppressant are not administered simultaneously with the therapeutic macromolecule.

In one embodiment of any one of the methods provided, the synthetic nanocarriers comprising the immunosuppressant are not administered concomitantly with a viral vector or are administered concomitantly with a combination of a viral vector and a separate administration (e.g., not in the same administered composition and/or administered separately for a different purpose such as not for inducing or increasing autophagy) of synthetic nanocarriers comprising an immunosuppressant. In one embodiment of any one of the methods provided, the synthetic nanocarriers comprising the immunosuppressant are not administered simultaneously with the viral vector.

In one embodiment of any one of the methods provided, the synthetic nanocarriers comprising the immunosuppressant are not administered concomitantly with an APC presentable antigen or are administered concomitantly with a combination of an APC presentable antigen and a separate administration (e.g., not in the same administered composition and/or administered separately for a different purpose such as not for inducing or increasing autophagy) of synthetic nanocarriers comprising an immunosuppressant. In one embodiment of any one of the methods provided, the synthetic nanocarriers comprising the immunosuppressant are not administered simultaneously with the APC presentable antigen.

In one embodiment of any one of the methods provided, the method further comprises providing the subject needing the induction or increase in autophagy or having or suspected of having the autophagy-associated disease or disorder.

In one embodiment of any one of the methods provided herein, the method further comprises identifying the subject as being in need of a method provided herein or as needing the induction or increase in autophagy or having or at risk of having an autophagy-associated disease or disorder.

In one embodiment of any one of the methods provided herein, the synthetic nanocarriers comprising an immunosuppressant for inducing or increasing autophagy is in an effective amount for inducing or increasing autophagy in a subject. In one embodiment of any one of the methods provided herein, the synthetic nanocarriers comprising an immunosuppressant for treating or preventing an autophagy-associated disease or disorder is in an effective amount for treating or preventing the autophagy-associated disease or disorder. The method may include a separate administration of synthetic nanocarriers comprising an immunosuppressant for a different purpose (e.g., not for inducing or increasing autophagy), and in such embodiments, the synthetic nanocarriers comprising an immunosuppressant are administered in an amount effective for such different purpose.

In one embodiment of any one of the methods provided herein, the autophagy-associated disease or disorder is a liver disease.

In one embodiment of any one of the methods provided, the subject is any one of the subjects provided herein. In one embodiment, the subject is a pediatric or a juvenile subject.

In one embodiment of any one of the methods provided, the immunosuppressant is an mTOR inhibitor. In one embodiment of any one of the methods provided, the mTOR inhibitor is rapamycin or a rapalog.

In one embodiment of any one of the methods provided, the immunosuppressant is encapsulated in the synthetic nanocarriers.

In one embodiment of any one of the methods provided, the synthetic nanocarriers comprise lipid nanoparticles, polymeric nanoparticles, metallic nanoparticles, surfactant-based emulsions, dendrimers, buckyballs, nanowires, virus-like particles or peptide or protein particles. In one embodiment of any one of the methods provided, the polymeric nanoparticles comprise a polyester, polyester attached to a polyether, polyamino acid, polycarbonate, polyacetal, polyketal, polysaccharide, polyethyloxazoline or polyethyleneimine. In one embodiment of any one of the methods provided, the polymeric nanoparticles comprise a polyester or a polyester attached to a polyether. In one embodiment of any one of the methods provided, the polyester comprises a poly(lactic acid), poly(glycolic acid), poly(lactic-co-glycolic acid) or polycaprolactone. In one embodiment of any one of the methods provided, the polymeric nanoparticles comprise a polyester and a polyester attached to a polyether. In one embodiment of any one of the methods provided, the polyether comprises polyethylene glycol or polypropylene glycol.

In one embodiment of any one of the methods provided, the mean of a particle size distribution obtained using dynamic light scattering of a population of the synthetic nanocarriers is a diameter greater than 110 nm, greater than 150 nm, greater than 200 nm, or greater than 250 nm. In one embodiment of any one of the methods provided, the mean of a particle size distribution obtained using dynamic light scattering of a population of the synthetic nanocarriers is less than 5 μm, less than 4 μm, less than 3 μm, less than 2 μm, less than 1 μm, less than 750 nm, less than 500 nm, less than 450 nm, less than 400 nm, less than 350 nm, or less than 300 nm.

In one embodiment of any one of the methods provided, the load of immunosuppressant comprised in the synthetic nanocarriers, on average across the synthetic nanocarriers, is between 0.1% and 50% (weight/weight), between 4% and 40%, between 5% and 30%, or between 8% and 25%.

In one embodiment of any one of the methods provided, an aspect ratio of a population of the synthetic nanocarriers is greater than or equal to 1:1, 1:1.2, 1:1.5, 1:2, 1:3, 1:5, 1:7 or 1:10.

In one embodiment of any one of the methods provided herein, the subject is one that has a liver disease or disorder and/or is in need of the compositions provided herein for treating or preventing a liver disease or disorder or liver toxicity.

In one embodiment of any one of the methods provided herein, the subject is one that does not have a liver disease or disorder and/or is not one in need of the compositions provided herein for treating or preventing a liver disease or disorder or liver toxicity. In one embodiment of any one of the methods provided herein, the subject is one that does not have inborn errors of metabolism. In one embodiment of any one of the methods provided herein, the subject is one that does not have an organic acidemia. In one embodiment of any one of the methods provided herein, the subject is one that does not have methylmalonic acidemia or ornithine decarboxylase deficiency.

In another aspect, a composition as described in any one of the methods provided or any one of the Examples is provided. In one embodiment, the composition is any one of the compositions for administration according to any one of the methods provided.

In another aspect, any one of the compositions is for use in any one of the methods provided.

### BRIEF DESCRIPTION OF THE FIGURES

FIG. 1 shows that preventative or therapeutic treatment with ImmTOR™ decreases serum levels of alanine aminotransferase (ALT) at 24 hours after mouse challenge with a polyclonal T cell activator, concanavalin A (Con A). Statistical significance is indicated (*, p<0.05).

FIG. 2 shows levels of urinary orotic acid in a murine model of OTC deficiency after administration of 4, 8, 12 mg/kg ImmTOR™, 1E13/kg AAV-OTC, or empty nanoparticles as a negative control.

FIG. 3 shows preventive or therapeutic treatment with ImmTOR™ decreases serum ALT at 24 hours after mouse challenge with acetaminophen (APAP). Statistical significance indicated (* p<0.05).

FIGS. 4A-4F show the results of a tolerability study of ImmTOR™ nanoparticles in juvenile OTC^{spf-ash} mice. FIG. 4A shows that EMPTY-nanoparticles or ImmTOR™ nanoparticles were i.v. injected in OTC^{spf-ash} juvenile mice. Injected mice were tested for: ALT and AST (FIG. 4B), body weight (FIG. 4C), plasma Ammonia levels (FIG. 4D), Urinary Orotic acid (FIG. 4E), and autophagy markers in liver lysates of treated mice (FIG. 4F).

FIGS. 5A-5D show the results of a tolerability study of ImmTOR™ nanoparticles in juvenile OTCspf-ash mice intravenously injected with 4, 8, or 12 mg/kg ImmTOR™ nanoparticles or 12 mg/kg of empty-particles (n=3/group). FIG. 5A shows urinary orotic acid levels quantified 2, 7, and 14 days post-injection. FIG. 5B shows body weights of the mice 2, 7, and 14 days post-injection. FIGS. 5C and 5D show levels of aspartate aminotransferase (AST) and alanine aminotransferase (ALT) activity, respectively.

FIGS. 6A-6D show the results of a tolerability study of ImmTOR™ nanoparticles in juvenile OTCspf-ash mice intravenously injected with 12 mg/kg ImmTOR™ nanoparticles or 12 mg/kg of empty-particles (n=4/group). FIG. 6A illustrates the protocol. FIG. 6B shows urinary orotic acid levels at 2, 7, and 14 days post-injection. FIG. 6C depicts the urinary orotic acid level at 14 days post-infection. FIG. 6D shows hepatic ammonia levels at 14 days post-injection. Statistical analysis was performed by one-way ANOVA with Tukey's multiple comparison test. (*p-value<0.05, ***p-value<0.0001).

FIGS. 7A-7B show ImmTOR™ particles induce autophagy in the liver in juvenile OTCspf-ash mice intravenously injected with 12 mg/kg ImmTOR™ nanoparticles or 12 mg/kg of empty-particles (n=4/group). FIG. 7A shows a Western blot analysis of ATG7, LC3II, and p62. FIG. 7B shows densiometric quantifications for the levels of ATG7, LC3II, and p62. Statistical analysis was performed by one-way ANOVA with Tukey's multiple comparison test. (*p-value<0.05).

### DETAILED DESCRIPTION OF THE INVENTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes.

As used in this specification and the appended claims, the singular forms “a,” “an” and “the” include plural referents unless the content clearly dictates otherwise. For example, reference to “a polymer” includes a mixture of two or more such molecules or a mixture of differing molecular weights of a single polymer species, reference to “a synthetic nanocarrier” includes a mixture of two or more such synthetic nanocarriers or a plurality of such synthetic nanocarriers, and the like.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, elements, characteristics, properties, method/process steps or limitations) but not the exclusion of any other integer or group of integers. Thus, as used herein, the term “comprising” is inclusive and does not exclude additional, unrecited integers or method/process steps.

In embodiments of any one of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, elements, characteristics, properties, method/process steps or limitations) alone.

### A. Introduction

Autophagy is one of the mechanisms by which components are degraded within a cell. It is a global term for a system in which components present in the cytoplasm are moved to an autophagosome (lysosome), which is a digestive organelle, and are degraded. It is believed that induction of autophagy can inhibit inflammation and otherwise prevent and treat diseases and disorders via known effects of autophagy such as organelle degradation, intracellular purification, and antigen presentation.

As provided herein, it has been found that administration of synthetic nanocarriers comprising an immunosuppressant (e.g., rapamycin) induces autophagy when administered. As described herein, the inventors surprisingly found that compositions comprising synthetic nanocarriers comprising an immunosuppressant can have beneficial effects on liver toxicity and diseases and disorders so associated. Without being bound by theory, it is believed that these effects are achieved, at least in part, due to an increase in autophagy in the liver.

Thus, provided herein are methods, and related compositions, for treating a subject with an autophagy-associated disease or disorder, for example, by administering synthetic nanocarriers comprising an immunosuppressant. As demonstrated herein, such methods and compositions were found to alter biomarkers consistent with an increase autophagy, such as in models of liver disease. Said compositions can be efficacious when administered in the absence of other therapies or can be efficacious as provided herein in combination with other therapies. The compositions described herein can also be useful to complement existing therapies, such as gene therapies, even when not administered concomitantly.

The invention will now be described in more detail below.

### B. Definitions

“Administering” or “administration” or “administer” means giving a material to a subject in a manner such that there is a pharmacological result in the subject. This may be direct or indirect administration, such as by inducing or directing another subject, including another clinician or the subject itself, to perform the administration.

“Amount effective” in the context of a composition or dose for administration to a subject refers to an amount of the composition or dose that produces one or more desired responses in the subject, e.g., inducing or increasing autophagy or preventing or treating a disease or disorder mediated by autophagy as is described herein. Therefore, in some embodiments, an amount effective is any amount of a composition or dose provided herein that produces one or more of the desired therapeutic effects and/or preventative responses as provided herein. This amount can be for in vitro or in vivo purposes. For in vivo purposes, the amount can be one that a clinician would believe may have a clinical benefit for a subject in need thereof. Any one of the compositions or doses, including label doses, as provided herein can be in an amount effective.

Amounts effective can involve reducing the level of an undesired response, although in some embodiments, it involves preventing an undesired response altogether. Amounts effective can also involve delaying the occurrence of an undesired response. An amount that is effective can also be an amount that produces a desired therapeutic endpoint or a desired therapeutic result. In other embodiments, the amounts effective can involve enhancing the level of a desired response, such as a therapeutic endpoint or result. Amounts effective, preferably, result in a preventative result or therapeutic result or endpoint with respect to an autophagy-associated disease or disorder in any one of the subjects provided herein. The achievement of any of the foregoing can be monitored by routine methods.

Amounts effective will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation. It is generally preferred that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reason.

“APC presentable antigen” means an antigen that can be presented for recognition by cells of the immune system, such as presented by antigen presenting cells, including but not limited to dendritic cells, B cells or macrophages. The APC presentable antigen can be presented for recognition by cells, such as recognition by T cells. Such antigens are recognized by and trigger an immune response in a T cell via presentation of the antigen or portion thereof bound to a Class I or Class II major histocompatibility complex molecule (MHC), or bound to a CD1 complex.

“Assessing a therapeutic or preventative response” refers to any measurement or determination of the level, presence or absence, reduction in, increase in, etc. of a therapeutic or preventative response in vitro or in vivo. Such measurements or determinations may be performed on one or more samples obtained from a subject. Such assessing can be performed with any of the methods provided herein or otherwise known in the art. The assessing may be assessing any one or more of the biomarkers provided herein or otherwise known in the art. For example, the assessing may be assessing any one or more markers of autophagy or any one of the autophagy-associated diseases or disorders provided herein or otherwise known in the art. In one embodiment, the marker(s) can be of liver disease.

With respect to liver disease, aspartate aminotransferase (AST) levels, alkaline phosphatase (ALP), gamma-glutamyl transpeptidase (GGT), bilirubin, prothrombin time, total protein, globulin, prothrombin, and/or albumin may be assessed.

In some embodiments, the markers of inflammation are cytokines/chemokines, immune-related effectors, acute phase proteins (e.g., C-reactive protein, serum amyloid A), reactive oxygen and nitrogen species, prostaglandins, and cyclooxygenase-related factors (e.g., transcription factors, growth factors).

“Attach” or “Attached” or “Couple” or “Coupled” (and the like) means to chemically associate one entity (for example a moiety) with another. In some embodiments, the attaching is covalent, meaning that the attachment occurs in the context of the presence of a covalent bond between the two entities. In non-covalent embodiments, the non-covalent attaching is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In embodiments, encapsulation is a form of attaching or coupling.

“Autophagy-associated disease” or “autophagy-associated disorder” refers to a disease or disorder that is caused by a disruption in autophagy or cellular self-digestion or for which there would be a benefit from the induction or increase in autophagy.

“Average” refers to the mean unless indicated otherwise.

“Concomitantly” means administering two or more materials/agents to a subject in a manner that is correlated in time, preferably sufficiently correlated in time such that a first composition (e.g., synthetic nanocarriers comprising an immunosuppressant) has an effect on a second composition, such as increasing the efficacy of the second composition, preferably the two or more materials/agents are administered in combination. In embodiments, concomitant administration may encompass administration of two or more compositions within a specified period of time. In some embodiments, the two or more compositions are administered within 1 month, within 1 week, within 1 day, or within 1 hour. In some embodiments, concomitant administration encompasses simultaneous administration of two or more compositions. In some embodiments, when two or more compositions are not administered concomitantly, there is little to no effect of the first composition (e.g., synthetic nanocarriers comprising an immunosuppressant) on the second composition. In one embodiment of any one of the methods provided herein, the synthetic nanocarriers comprising an immunosuppressant for inducing or increasing autophagy or treating or preventing an autophagy-associated disease or disorder is not administered to effect a second composition (e.g., not to effect an immune response, such as an antibody response, against the second composition), such as a different therapeutic, such as a therapeutic macromolecule, viral vector, APC presentable antigen, etc.

“Dosage form” means a pharmacologically and/or immunologically active material in a medium, carrier, vehicle, or device suitable for administration to a subject. Any one of the compositions or doses provided herein may be in a dosage form.

“Dose” refers to a specific quantity of a pharmacologically and/or immunologically active material for administration to a subject for a given time. Unless otherwise specified, the doses recited for compositions comprising synthetic nanocarriers comprising an immunosuppressant refer to the weight of the immunosuppressant (i.e., without the weight of the synthetic nanocarrier material). When referring to a dose for administration, in an embodiment of any one of the methods, compositions or kits provided herein, any one of the doses provided herein is the dose as it appears on a label/label dose.

“Encapsulate” means to enclose at least a portion of a substance within a synthetic nanocarrier. In some embodiments, a substance is enclosed completely within a synthetic nanocarrier. In other embodiments, most or all of a substance that is encapsulated is not exposed to the local environment external to the synthetic nanocarrier. In other embodiments, no more than 50%, 40%, 30%, 20%, 10% or 5% (weight/weight) is exposed to the local environment. Encapsulation is distinct from absorption, which places most or all of a substance on a surface of a synthetic nanocarrier, and leaves the substance exposed to the local environment external to the synthetic nanocarrier. In embodiments of any one of the methods or compositions provided herein, the immunosuppressants are encapsulated within the synthetic nanocarriers.

“Identifying a subject” is any action or set of actions that allows a clinician to recognize a subject as one who may benefit from the methods or compositions provided herein or some other indicator as provided. Preferably, the identified subject is one who is in need of autophagy induction or increase or preventative or therapeutic treatment for an autophagy-associated disease or disorder. Such subjects include any subject that has or is at risk of having an autophagy-associated disease or disorder. In some embodiments, the subject is suspected of having or determined to have a likelihood or risk of having an autophagy-associated disease or disorder based on symptoms (and/or lack thereof), patterns of behavior (e.g., that would put a subject at risk), and/or based on one or more tests described herein (e.g., biomarker assays).

In one embodiment of any one of the methods provided herein, the method further comprises identifying a subject in need of a composition or method as provided herein. The action or set of actions may be either directly oneself or indirectly, such as, but not limited to, an unrelated third party that takes an action through reliance on one's words or deeds.

“Immunosuppressant” means a compound that can cause a tolerogenic effect through its effects on APCs. A tolerogenic effect generally refers to the modulation by the APC or other immune cells that reduces, inhibits or prevents an undesired immune response to an antigen in a durable fashion. In one embodiment of any one of the methods or compositions provided, the immunosuppressant is one that causes an APC to promote a regulatory phenotype in one or more immune effector cells. For example, the regulatory phenotype may be characterized by the inhibition of the production, induction, stimulation or recruitment of antigen-specific CD4+ T cells or B cells, the inhibition of the production of antigen-specific antibodies, the production, induction, stimulation or recruitment of Treg cells (e.g., CD4+CD25highFoxP3+ Treg cells), etc. This may be the result of the conversion of CD4+ T cells or B cells to a regulatory phenotype. This may also be the result of induction of FoxP3 in other immune cells, such as CD8+ T cells, macrophages and iNKT cells. In one embodiment of any one of the methods or compositions provided, the immunosuppressant is one that affects the response of the APC after it processes an antigen. In another embodiment of any one of the methods or compositions provided, the immunosuppressant is not one that interferes with the processing of the antigen. In a further embodiment of any one of the methods or compositions provided, the immunosuppressant is not an apoptotic-signaling molecule. In another embodiment of any one of the methods or compositions provided, the immunosuppressant is not a phospholipid.

Immunosuppressants include, but are not limited to mTOR inhibitors, such as rapamycin or a rapamycin analog (i.e., rapalog); TGF-β signaling agents; TGF-β receptor agonists; histone deacetylase inhibitors, such as Trichostatin A; corticosteroids; inhibitors of mitochondrial function, such as rotenone; P38 inhibitors; NF-κβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; proteasome inhibitors; kinase inhibitors; etc. “Rapalog”, as used herein, refers to a molecule that is structurally related to (an analog) of rapamycin (sirolimus). Examples of rapalogs include, without limitation, temsirolimus (CCI-779), everolimus (RAD001), ridaforolimus (AP-23573), and zotarolimus (ABT-578). Additional examples of rapalogs may be found, for example, in WO Publication WO 1998/002441 and U.S. Pat. No. 8,455,510, the rapalogs of which are incorporated herein by reference in their entirety. Further immunosuppressants are known to those of skill in the art, and the invention is not limited in this respect.

In embodiments, when coupled to the synthetic nanocarriers, the immunosuppressant is an element that is in addition to the material that makes up the structure of the synthetic nanocarrier. For example, in one such embodiment, where the synthetic nanocarrier is made up of one or more polymers, the immunosuppressant is a compound that is in addition and coupled to the one or more polymers. As another example, in one such embodiment, where the synthetic nanocarrier is made up of one or more lipids, the immunosuppressant is again in addition and coupled to the one or more lipids.

“Increasing autophagy” or the like means increasing the level of autophagy in the subject relative to a control. In some embodiments, autophagy is increased, e.g., is increased at least 20-40%, more preferably by at least 50-75%, and most preferably by more than 80% relative to a control. Preferably, the increase is at least two-fold. In some embodiments, the control is autophagy activity (e.g., from the liver) from the same subject at a prior period in time. In some embodiments, the control autophagy level is from an untreated subject having the same autophagy-associated disease or disorder. In some embodiments, a control is an average level of autophagy in a population of untreated subjects having the same autophagy-associated disease or disorder.

In some embodiments, increasing autophagy comprises modulating the levels of one or more markers of autophagy. In some embodiments, the marker is increased or decreased by at least 20-40%, more preferably by at least 50-75%, and most preferably by more than 80% relative to a control. Preferably the increase or decrease is at least two-fold. “Markers of autophagy” are those which usually indicate autophagy in the subject (e.g., in the liver of the subject). They can be determined with methods known to one of skill in the art such as in cells, tissues or body fluids from the subject, in particular from a liver biopsy or in the blood serum or blood plasma of the subject. Markers of autophagy include, for example, LC3II, p62, and ATG7.

“Load”, when coupled to a synthetic nanocarrier, is the amount of the immunosuppressant coupled to the synthetic nanocarrier based on the total dry recipe weight of materials in an entire synthetic nanocarrier (weight/weight). Generally, such a load is calculated as an average across a population of synthetic nanocarriers. In one embodiment of any one of the methods or compositions provided, the load on average across the synthetic nanocarriers is between 0.1% and 50%. In another of any one of the methods or compositions provided, the load on average across the synthetic nanocarriers is between 4%, 5%, 65, 7%, 8% or 9% and 40% or between 4%, 5%, 65, 7%, 8% or 9% and 30%. In another of any one of the methods or compositions provided, the load on average across the synthetic nanocarriers is between 10% and 40% or between 10% and 30%. In another embodiment of any one of the methods or compositions provided, the load is between 0.1% and 20%. In a further embodiment of any one of the methods or compositions provided, the load is between 0.1% and 10%. In still a further embodiment of any one of the methods or compositions provided, the load is between 1% and 10%. In still a further embodiment of any one of the methods or compositions provided, the load is between 7% and 20%. In yet another embodiment of any one of the methods or compositions provided, the load is at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least at least 7%, at least 8%, at least 9%, at least 10%, at least 11%, at least 12%, at least 13%, at least 14%, at least 15%, at least 16%, at least 17%, at least 18%, at least 19% at least 20%, at least 21%, at least 22%, at least 23%, at least 24%, at least 25%, at least 26%, at least 27%, at least 28%, at least 29% or at least 30% on average across the population of synthetic nanocarriers. In yet a further embodiment of any one of the methods or compositions provided, the load is 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% on average across the population of synthetic nanocarriers. In some embodiments of any one of the above embodiments, the load is no more than 35%, 30% or 25% on average across a population of synthetic nanocarriers. In any one of the methods, compositions or kits provided herein, the load of the immunosuppressant, such as rapamycin, may be any one of the loads provided herein. In embodiments of any one of the methods or compositions provided, the load is calculated as known in the art.

In some embodiments, the immunosuppressant load of the nanocarrier in suspension is calculated by dividing the immunosuppressant content of the nanocarrier as determined by HPLC analysis of the test article by the nanocarrier mass. The total polymer content is measured either by gravimetric yield of the dry nanocarrier mass or by the determination of the nanocarrier solution total organic content following pharmacopeia methods and corrected for PVA content.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheroidal synthetic nanocarrier, the maximum and minimum dimension of a synthetic nanocarrier would be substantially identical, and would be the size of its diameter. Similarly, for a cuboidal synthetic nanocarrier, the minimum dimension of a synthetic nanocarrier would be the smallest of its height, width or length, while the maximum dimension of a synthetic nanocarrier would be the largest of its height, width or length. In an embodiment, a minimum dimension of at least 75%, preferably at least 80%, more preferably at least 90%, of the synthetic nanocarriers in a sample, based on the total number of synthetic nanocarriers in the sample, is equal to or greater than 100 nm. In an embodiment, a maximum dimension of at least 75%, preferably at least 80%, more preferably at least 90%, of the synthetic nanocarriers in a sample, based on the total number of synthetic nanocarriers in the sample, is equal to or less than 5 μm. Preferably, a minimum dimension of at least 75%, preferably at least 80%, more preferably at least 90%, of the synthetic nanocarriers in a sample, based on the total number of synthetic nanocarriers in the sample, is greater than 110 nm, more preferably greater than 120 nm, more preferably greater than 130 nm, and more preferably still greater than 150 nm. Aspects ratios of the maximum and minimum dimensions of inventive synthetic nanocarriers may vary depending on the embodiment. For instance, aspect ratios of the maximum to minimum dimensions of the synthetic nanocarriers may vary from 1:1 to 1,000,000:1, preferably from 1:1 to 100,000:1, more preferably from 1:1 to 10,000:1, more preferably from 1:1 to 1000:1, still more preferably from 1:1 to 100:1, and yet more preferably from 1:1 to 10:1.

Preferably, a maximum dimension of at least 75%, preferably at least 80%, more preferably at least 90%, of the synthetic nanocarriers in a sample, based on the total number of synthetic nanocarriers in the sample is equal to or less than 3 μm, more preferably equal to or less than 2 μm, more preferably equal to or less than 1 μm, more preferably equal to or less than 800 nm, more preferably equal to or less than 600 nm, and more preferably still equal to or less than 500 nm. In preferred embodiments, a minimum dimension of at least 75%, preferably at least 80%, more preferably at least 90%, of the synthetic nanocarriers in a sample, based on the total number of synthetic nanocarriers in the sample, is equal to or greater than 100 nm, more preferably equal to or greater than 120 nm, more preferably equal to or greater than 130 nm, more preferably equal to or greater than 140 nm, and more preferably still equal to or greater than 150 nm. Measurement of synthetic nanocarrier dimensions (e.g., diameter) may be obtained by suspending the synthetic nanocarriers in a liquid (usually aqueous) media and using dynamic light scattering (DLS) (e.g. using a Brookhaven ZetaPALS instrument). For example, a suspension of synthetic nanocarriers can be diluted from an aqueous buffer into purified water to achieve a final synthetic nanocarrier suspension concentration of approximately 0.01 to 0.1 mg/mL. The diluted suspension may be prepared directly inside, or transferred to, a suitable cuvette for DLS analysis. The cuvette may then be placed in the DLS, allowed to equilibrate to the controlled temperature, and then scanned for sufficient time to acquire a stable and reproducible distribution based on appropriate inputs for viscosity of the medium and refractive indicies of the sample. The effective diameter, or mean of the distribution, can then reported. “Dimension” or “size” or “diameter” of synthetic nanocarriers means the mean of a particle size distribution obtained using dynamic light scattering in some embodiments.

“Pharmaceutically acceptable excipient” or “pharmaceutically acceptable carrier” means a pharmacologically inactive material used together with a pharmacologically active material to formulate the compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers. Any one of the compositions provided herein may include a pharmaceutically acceptable excipient or carrier.

“Protocol” refers to any dosing regimen of one or more substances to a subject. A dosing regimen may include the amount, frequency, rate, duration and/or mode of administration. In some embodiments, such a protocol may be used to administer one or more compositions of the invention to one or more test subjects. Therapeutic/preventative responses in these test subjects can then be assessed to determine whether or not the protocol was effective in generating a desired response, such as prevention and/or treatment of an autophagy-associated disease or disorder, or the induction or an increase in autophagy. Whether or not a protocol had a desired effect can be determined using any of the methods provided herein or otherwise known in the art. For example, a population of cells may be obtained from a subject to which a composition provided herein has been administered according to a specific protocol in order to determine whether or not specific enzymes, biomarkers, etc. were generated, activated, etc. Useful methods for detecting the presence and/or number of biomarkers include, but are not limited to, flow cytometric methods (e.g., FACS) and immunohistochemistry methods. Antibodies and other binding agents for specific staining of certain biomarkers, are commercially available. Such kits typically include staining reagents for multiple antigens that allow for FACS-based detection, separation and/or quantitation of a desired cell population from a heterogeneous population of cells. Any one of the methods provided herein can include a step of determining a protocol and/or the administering is done based on a protocol determined to have any one of the beneficial results or desired beneficial result as provided herein, such as inducing or increasing autophagy.

“Providing a subject” is any action or set of actions that causes a clinician to come in contact with a subject and administer a composition provided herein thereto or to perform a method provided herein thereupon. Preferably, the subject is one who is in need of autophagy induction or increase or the prevention or treatment of an autophagy-associated disease or disorder, etc. The action or set of actions may be taken either directly oneself or indirectly. In one embodiment of any one of the methods provided herein, the method further comprises providing a subject.

“Repeat dose” or “repeat dosing” or the like means at least one additional dose or dosing that is administered to a subject subsequent to an earlier dose or dosing of the same material. For example, a repeated dose of a nanocarrier comprising an immunosuppressant after a prior dose of the same material. While the material may be the same, the amount of the material in the repeated dose may be different from the earlier dose. A repeat dose may be administered as provided herein. Repeat dosing is considered to be efficacious if it results in a beneficial effect for the subject. Preferably, efficacious repeat dosing results in increased autophagy. Any one of the methods provided herein can include a step of repeat dosing.

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like. In any one of the methods, compositions and kits provided herein, the subject is human.

“Synthetic nanocarrier(s)” means a discrete object that is not found in nature, and that possesses at least one dimension that is less than or equal to 5 microns in size. Synthetic nanocarriers may be a variety of different shapes, including but not limited to spheroidal, cuboidal, pyramidal, oblong, cylindrical, toroidal, and the like. Synthetic nanocarriers comprise one or more surfaces.

A synthetic nanocarrier can be, but is not limited to, one or a plurality of lipid-based nanoparticles (also referred to herein as lipid nanoparticles, i.e., nanoparticles where the majority of the material that makes up their structure are lipids), polymeric nanoparticles, metallic nanoparticles, surfactant-based emulsions, dendrimers, buckyballs, nanowires, virus-like particles (i.e., particles that are primarily made up of viral structural proteins but that are not infectious or have low infectivity), peptide or protein-based particles (also referred to herein as protein particles, i.e., particles where the majority of the material that makes up their structure are peptides or proteins) (such as albumin nanoparticles) and/or nanoparticles that are developed using a combination of nanomaterials such as lipid-polymer nanoparticles. Synthetic nanocarriers may be a variety of different shapes, including but not limited to spheroidal, cuboidal, pyramidal, oblong, cylindrical, toroidal, and the like. Examples of synthetic nanocarriers include (1) the biodegradable nanoparticles disclosed in U.S. Pat. No. 5,543,158 to Gref et al., (2) the polymeric nanoparticles of Published US Patent Application 20060002852 to Saltzman et al., (3) the lithographically constructed nanoparticles of Published US Patent Application 20090028910 to DeSimone et al., (4) the disclosure of WO 2009/051837 to von Andrian et al., (5) the nanoparticles disclosed in Published US Patent Application 2008/0145441 to Penades et al., (6) the nanoprecipitated nanoparticles disclosed in P. Paolicelli et al., “Surface-modified PLGA-based Nanoparticles that can Efficiently Associate and Deliver Virus-like Particles” Nanomedicine. 5(6):843-853 (2010), and (7) those of Look et al., Nanogel-based delivery of mycophenolic acid ameliorates systemic lupus erythematosus in mice” J. Clinical Investigation 123(4):1741-1749(2013).

Synthetic nanocarriers may have a minimum dimension of equal to or less than about 100 nm, preferably equal to or less than 100 nm, do not comprise a surface with hydroxyl groups that activate complement or alternatively comprise a surface that consists essentially of moieties that are not hydroxyl groups that activate complement in some embodiments. In an embodiment, synthetic nanocarriers that have a minimum dimension of equal to or less than about 100 nm, preferably equal to or less than 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than 100 nm, do not comprise a surface that activates complement or alternatively comprise a surface that consists essentially of moieties that do not activate complement. In embodiments, synthetic nanocarriers exclude virus-like particles. In embodiments, synthetic nanocarriers may possess an aspect ratio greater than or equal to 1:1, 1:1.2, 1:1.5, 1:2, 1:3, 1:5, 1:7, or greater than 1:10.

A “therapeutic macromolecule” refers to any protein, carbohydrate, lipid or nucleic acid that may be administered to a subject and have a therapeutic effect. In some embodiments, the therapeutic macromolecule may be a therapeutic polynucleotide or therapeutic protein.

“Therapeutic polynucleotide” means any polynucleotide or polynucleotide-based therapy that may be administered to a subject and have a therapeutic effect. Such therapies include gene silencing. Examples of such therapy are known in the art, and include, but are not limited to, naked RNA (including messenger RNA, modified messenger RNA, and forms of RNAi).

“Therapeutic protein” means any protein or protein-based therapy that may be administered to a subject and have a therapeutic effect. Such therapies include protein replacement and protein supplementation therapies. Such therapies also include the administration of exogenous or foreign proteins, antibody therapies, etc. Therapeutic proteins comprise, but are not limited to, enzymes, enzyme cofactors, hormones, blood clotting factors, cytokines, growth factors, monoclonal antibodies, antibody-drug conjugates, and polyclonal antibodies.

“Treating” refers to the administration of one or more therapeutics with the expectation that the subject may have a resulting benefit due to the administration. Treating may be direct or indirect, such as by inducing or directing another subject, including another clinician or the subject itself, to treat the subject.

“Viral vector” means a vector construct with viral components, such as capsid and/or coat proteins, that has been adapted to comprise and deliver a transgene or nucleic acid material, such as one that encodes a therapeutic, such as a therapeutic protein, which transgene or nucleic acid material may be expressed as provided herein.

### C. Methods and Related Compositions

Provided herein are methods and related compositions useful for inducing or increasing autophagy and/or treating and/or preventing autophagy-associated diseases and disorders, e.g., by inducing or increasing autophagy. The methods and compositions advantageously provide a therapeutic that prevents and/or treats a variety of autophagy-mediated diseases and disorders, e.g., by inducing or increasing autophagy, and does not necessarily require a disease-specific treatment, although a disease-specific treatment may also be provided to the subject.

### Synthetic Nanocarriers

A wide variety of synthetic nanocarriers can be used according to the invention. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

In some embodiments, it is desirable to use a population of synthetic nanocarriers that is relatively uniform in terms of size or shape so that each synthetic nanocarrier has similar properties. For example, at least 80%, at least 90%, or at least 95% of the synthetic nanocarriers of any one of the compositions or methods provided, based on the total number of synthetic nanocarriers, may have a minimum dimension or maximum dimension that falls within 5%, 10%, or 20% of the average diameter or average dimension of the synthetic nanocarriers.

Synthetic nanocarriers can be solid or hollow and can comprise one or more layers. In some embodiments, each layer has a unique composition and unique properties relative to the other layer(s). To give but one example, synthetic nanocarriers may have a core/shell structure, wherein the core is one layer (e.g. a polymeric core) and the shell is a second layer (e.g. a lipid bilayer or monolayer). Synthetic nanocarriers may comprise a plurality of different layers.

In some embodiments, synthetic nanocarriers may optionally comprise one or more lipids. In some embodiments, a synthetic nanocarrier may comprise a liposome. In some embodiments, a synthetic nanocarrier may comprise a lipid bilayer. In some embodiments, a synthetic nanocarrier may comprise a lipid monolayer. In some embodiments, a synthetic nanocarrier may comprise a micelle. In some embodiments, a synthetic nanocarrier may comprise a core comprising a polymeric matrix surrounded by a lipid layer (e.g., lipid bilayer, lipid monolayer, etc.). In some embodiments, a synthetic nanocarrier may comprise a non-polymeric core (e.g., metal particle, quantum dot, ceramic particle, bone particle, viral particle, proteins, nucleic acids, carbohydrates, etc.) surrounded by a lipid layer (e.g., lipid bilayer, lipid monolayer, etc.).

In other embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

In some embodiments, synthetic nanocarriers may optionally comprise one or more amphiphilic entities. In some embodiments, an amphiphilic entity can promote the production of synthetic nanocarriers with increased stability, improved uniformity, or increased viscosity. In some embodiments, amphiphilic entities can be associated with the interior surface of a lipid membrane (e.g., lipid bilayer, lipid monolayer, etc.). Many amphiphilic entities known in the art are suitable for use in making synthetic nanocarriers in accordance with the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used in the production of synthetic nanocarriers to be used in accordance with the present invention.

In some embodiments, synthetic nanocarriers may optionally comprise one or more carbohydrates. Carbohydrates may be natural or synthetic. A carbohydrate may be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan. In embodiments, the synthetic nanocarriers do not comprise (or specifically exclude) carbohydrates, such as a polysaccharide. In certain embodiments, the carbohydrate may comprise a carbohydrate derivative such as a sugar alcohol, including but not limited to mannitol, sorbitol, xylitol, erythritol, maltitol, and lactitol.

In some embodiments, synthetic nanocarriers can comprise one or more polymers. In some embodiments, the synthetic nanocarriers comprise one or more polymers that is a non-methoxy-terminated, pluronic polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers are non-methoxy-terminated, pluronic polymers. In some embodiments, all of the polymers that make up the synthetic nanocarriers are non-methoxy-terminated, pluronic polymers. In some embodiments, the synthetic nanocarriers comprise one or more polymers that is a non-methoxy-terminated polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers are non-methoxy-terminated polymers. In some embodiments, all of the polymers that make up the synthetic nanocarriers are non-methoxy-terminated polymers. In some embodiments, the synthetic nanocarriers comprise one or more polymers that do not comprise pluronic polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers do not comprise pluronic polymer. In some embodiments, all of the polymers that make up the synthetic nanocarriers do not comprise pluronic polymer. In some embodiments, such a polymer can be surrounded by a coating layer (e.g., liposome, lipid monolayer, micelle, etc.). In some embodiments, elements of the synthetic nanocarriers can be attached to the polymer.

Immunosuppressants can be coupled to the synthetic nanocarriers by any of a number of methods. Generally, the attaching can be a result of bonding between the immunosuppressants and the synthetic nanocarriers. This bonding can result in the immunosuppressants being attached to the surface of the synthetic nanocarriers and/or contained (encapsulated) within the synthetic nanocarriers. In some embodiments of any one of the methods or compositions provided, however, the immunosuppressants are encapsulated by the synthetic nanocarriers as a result of the structure of the synthetic nanocarriers rather than bonding to the synthetic nanocarriers. In preferable embodiments of any one of the methods or compositions provided, the synthetic nanocarrier comprises a polymer as provided herein, and the immunosuppressants are coupled to the polymer.

When coupling occurs as a result of bonding between the immunosuppressants and synthetic nanocarriers, the coupling may occur via a coupling moiety. A coupling moiety can be any moiety through which an immunosuppressant is bonded to a synthetic nanocarrier. Such moieties include covalent bonds, such as an amide bond or ester bond, as well as separate molecules that bond (covalently or non-covalently) the immunosuppressant to the synthetic nanocarrier. Such molecules include linkers or polymers or a unit thereof. For example, the coupling moiety can comprise a charged polymer to which an immunosuppressant electrostatically binds. As another example, the coupling moiety can comprise a polymer or unit thereof to which it is covalently bonded.

In preferred embodiments of any one of the methods or compositions provided, the synthetic nanocarriers comprise a polymer as provided herein. These synthetic nanocarriers can be completely polymeric or they can be a mix of polymers and other materials.

In some embodiments of any one of the methods or compositions provided, the polymers of a synthetic nanocarrier associate to form a polymeric matrix. In some of these embodiments of any one of the methods or compositions provided, a component, such as an immunosuppressant, can be covalently associated with one or more polymers of the polymeric matrix. In some embodiments of any one of the methods or compositions provided, covalent association is mediated by a linker. In some embodiments of any one of the methods or compositions provided, a component can be non-covalently associated with one or more polymers of the polymeric matrix. For example, in some embodiments of any one of the methods or compositions provided, a component can be encapsulated within, surrounded by, and/or dispersed throughout a polymeric matrix. Alternatively or additionally, a component can be associated with one or more polymers of a polymeric matrix by hydrophobic interactions, charge interactions, van der Waals forces, etc. A wide variety of polymers and methods for forming polymeric matrices therefrom are known conventionally.

Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

In some embodiments, the polymer comprises a polyester, polycarbonate, polyamide, or polyether, or unit thereof. In other embodiments, the polymer comprises poly(ethylene glycol) (PEG), polypropylene glycol, poly(lactic acid), poly(glycolic acid), poly(lactic-co-glycolic acid), or a polycaprolactone, or unit thereof. In some embodiments, it is preferred that the polymer is biodegradable. Therefore, in these embodiments, it is preferred that if the polymer comprises a polyether, such as poly(ethylene glycol) or polypropylene glycol or unit thereof, the polymer comprises a block-co-polymer of a polyether and a biodegradable polymer such that the polymer is biodegradable. In other embodiments, the polymer does not solely comprise a polyether or unit thereof, such as poly(ethylene glycol) or polypropylene glycol or unit thereof.

Other examples of polymers suitable for use in the present invention include, but are not limited to polyethylenes, polycarbonates (e.g. poly(1,3-dioxan-2one)), polyanhydrides (e.g. poly(sebacic anhydride)), polypropylfumerates, polyamides (e.g. polycaprolactam), polyacetals, polyethers, polyesters (e.g., polylactide, polyglycolide, polylactide-co-glycolide, polycaprolactone, polyhydroxyacid (e.g. poly(β-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), poly(ethylene imine)-PEG copolymers.

In some embodiments, polymers in accordance with the present invention include polymers which have been approved for use in humans by the U.S. Food and Drug Administration (FDA) under 21 C.F.R. § 177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments, a synthetic nanocarrier comprising a hydrophilic polymeric matrix generates a hydrophilic environment within the synthetic nanocarrier. In some embodiments, polymers can be hydrophobic. In some embodiments, a synthetic nanocarrier comprising a hydrophobic polymeric matrix generates a hydrophobic environment within the synthetic nanocarrier. Selection of the hydrophilicity or hydrophobicity of the polymer may have an impact on the nature of materials that are incorporated within the synthetic nanocarrier.

In some embodiments, polymers may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). Certain embodiments may be made using the general teachings of U.S. Pat. No. 5,543,158 to Gref et al., or WO publication WO2009/051837 by Von Andrian et al.

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, poly(caprolactone), poly(caprolactone)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids. Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids. In embodiments, the synthetic nanocarriers may not comprise (or may exclude) cationic polymers.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and U.S. Pat. No. 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that the synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, synthetic nanocarriers do not comprise a polymeric component. In some embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

### Immunosuppressants

Any immunosuppressant as provided herein can be, in some embodiments of any one of the methods or compositions provided, coupled to synthetic nanocarriers. Immunosuppressants include, but are not limited to, statins; mTOR inhibitors, such as rapamycin or a rapamycin analog (rapalog); TGF-β signaling agents; TGF-β receptor agonists; histone deacetylase (HDAC) inhibitors; corticosteroids; inhibitors of mitochondrial function, such as rotenone; P38 inhibitors; NF-κβ inhibitors; adenosine receptor agonists; prostaglandin E2 agonists; phosphodiesterase inhibitors, such as phosphodiesterase 4 inhibitor; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors and oxidized ATPs. Immunosuppressants also include IDO, vitamin D3, cyclosporine A, aryl hydrocarbon receptor inhibitors, resveratrol, azathiopurine, 6-mercaptopurine, aspirin, niflumic acid, estriol, tripolide, interleukins (e.g., IL-1, IL-10), cyclosporine A, siRNAs targeting cytokines or cytokine receptors and the like.

Examples of statins include atorvastatin (LIPITOR®, TORVAST®), cerivastatin, fluvastatin (LESCOL®, LESCOL® XL), lovastatin (MEVACOR®, ALTOCOR®, ALTOPREV®), mevastatin (COMPACTIN®), pitavastatin (LIVALO®, PIAVA®), rosuvastatin (PRAVACHOL®, SELEKTINE®, LIPOSTAT®), rosuvastatin (CRESTOR®), and simvastatin (ZOCOR®, LIPEX®).

Examples of mTOR inhibitors include rapamycin and analogs thereof (e.g., CCL-779, RAD001, AP23573, C20-methallylrapamycin (C20-Marap), C16-(S)-butylsulfonamidorapamycin (C16-BSrap), C16-(S)-3-methylindolerapamycin (C16-iRap) (Bayle et al. Chemistry & Biology 2006, 13:99-107)), AZD8055, BEZ235 (NVP-BEZ235), chrysophanic acid (chrysophanol), deforolimus (MK-8669), everolimus (RAD0001), KU-0063794, PI-103, PP242, temsirolimus, and WYE-354 (available from Selleck, Houston, Tex., USA).

“Rapalog”, as used herein, refers to a molecule that is structurally related to (an analog) of rapamycin (sirolimus). Examples of rapalogs include, without limitation, temsirolimus (CCI-779), everolimus (RAD001), ridaforolimus (AP-23573), and zotarolimus (ABT-578). Additional examples of rapalogs may be found, for example, in WO Publication WO 1998/002441 and U.S. Pat. No. 8,455,510, the rapalogs of which are incorporated herein by reference in their entirety.

When coupled to a synthetic nanocarrier, the amount of the immunosuppressant coupled to the synthetic nanocarrier based on the total dry recipe weight of materials in an entire synthetic nanocarrier (weight/weight), is as described elsewhere herein. Preferably, in some embodiments of any one of the methods or compositions or kits provided herein, the load of the immunosuppressant, such as rapamycin or rapalog, is between 4%, 5%, 65, 7%, 8%, 9% or 10% and 25%, 26%, 27%, 28%, 29%, 30%, 31%, 32%, 33%, 34%, 35%, 36%, 37%, 38%, 39% or 40% by weight.

In regard to synthetic nanocarriers coupled to immunosuppressants, methods for coupling components to synthetic nanocarriers may be useful. Elements of the synthetic nanocarriers may be coupled to the overall synthetic nanocarrier, e.g., by one or more covalent bonds, or may be attached by means of one or more linkers. Additional methods of functionalizing synthetic nanocarriers may be adapted from Published US Patent Application 2006/0002852 to Saltzman et al., Published US Patent Application 2009/0028910 to DeSimone et al., or Published International Patent Application WO/2008/127532 A1 to Murthy et al.

In some embodiments, the coupling can be a covalent linker. In embodiments, immunosuppressants according to the invention can be covalently coupled to the external surface via a 1,2,3-triazole linker formed by the 1,3-dipolar cycloaddition reaction of azido groups with immunosuppressant containing an alkyne group or by the 1,3-dipolar cycloaddition reaction of alkynes with immunosuppressants containing an azido group. Such cycloaddition reactions are preferably performed in the presence of a Cu(I) catalyst along with a suitable Cu(I)-ligand and a reducing agent to reduce Cu(II) compound to catalytic active Cu(I) compound. This Cu(I)-catalyzed azide-alkyne cycloaddition (CuAAC) can also be referred as the click reaction.

Additionally, covalent coupling may comprise a covalent linker that comprises an amide linker, a disulfide linker, a thioether linker, a hydrazone linker, a hydrazide linker, an imine or oxime linker, an urea or thiourea linker, an amidine linker, an amine linker, and a sulfonamide linker.

Alternatively or additionally, synthetic nanocarriers can be coupled to components directly or indirectly via non-covalent interactions. In non-covalent embodiments, the non-covalent attaching is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. Such couplings may be arranged to be on an external surface or an internal surface of a synthetic nanocarrier. In embodiments of any one of the methods or compositions provided, encapsulation and/or absorption is a form of coupling.

For detailed descriptions of available conjugation methods, see Hermanson G T “Bioconjugate Techniques”, 2nd Edition Published by Academic Press, Inc., 2008. In addition to covalent attachment the component can be coupled by adsorption to a pre-formed synthetic nanocarrier or it can be coupled by encapsulation during the formation of the synthetic nanocarrier.

### D. Methods of Making and Using the Methods and Related Compositions

Synthetic nanocarriers may be prepared using a wide variety of methods known in the art. For example, synthetic nanocarriers can be formed by methods such as nanoprecipitation, flow focusing using fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755; U.S. Pat. Nos. 5,578,325 and 6,007,845; P. Paolicelli et al., “Surface-modified PLGA-based Nanoparticles that can Efficiently Associate and Deliver Virus-like Particles” Nanomedicine. 5(6):843-853 (2010)).

Materials may be encapsulated into synthetic nanocarriers as desirable using a variety of methods including but not limited to C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8— 21 (2006); P. Paolicelli et al., “Surface-modified PLGA-based Nanoparticles that can Efficiently Associate and Deliver Virus-like Particles” Nanomedicine. 5(6):843-853 (2010). Other methods suitable for encapsulating materials into synthetic nanocarriers may be used, including without limitation methods disclosed in U.S. Pat. No. 6,632,671 to Unger issued Oct. 14, 2003.

In certain embodiments, synthetic nanocarriers are prepared by a nanoprecipitation process or spray drying. Conditions used in preparing synthetic nanocarriers may be altered to yield particles of a desired size or property (e.g., hydrophobicity, hydrophilicity, external morphology, “stickiness,” shape, etc.). The method of preparing the synthetic nanocarriers and the conditions (e.g., solvent, temperature, concentration, air flow rate, etc.) used may depend on the materials to be attached to the synthetic nanocarriers and/or the composition of the polymer matrix.

If synthetic nanocarriers prepared by any of the above methods have a size range outside of the desired range, synthetic nanocarriers can be sized, for example, using a sieve.

Compositions provided herein may comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol).

Compositions according to the invention can comprise pharmaceutically acceptable excipients, such as preservatives, buffers, saline, or phosphate buffered saline. The compositions may be made using conventional pharmaceutical manufacturing and compounding techniques to arrive at useful dosage forms. In an embodiment of any one of the methods or compositions provided, compositions are suspended in sterile saline solution for injection together with a preservative. Techniques suitable for use in practicing the present invention may be found in Handbook of Industrial Mixing: Science and Practice, Edited by Edward L. Paul, Victor A. Atiemo-Obeng, and Suzanne M. Kresta, 2004 John Wiley & Sons, Inc.; and Pharmaceutics: The Science of Dosage Form Design, 2nd Ed. Edited by M. E. Auten, 2001, Churchill Livingstone. In an embodiment of any one of the methods or compositions provided, compositions are suspended in sterile saline solution for injection with a preservative.

It is to be understood that the compositions of the invention can be made in any suitable manner, and the invention is in no way limited to compositions that can be produced using the methods described herein. Selection of an appropriate method of manufacture may require attention to the properties of the particular moieties being associated.

In some embodiments of any one of the methods or compositions provided, compositions are manufactured under sterile conditions or are terminally sterilized. This can ensure that resulting compositions are sterile and non-infectious, thus improving safety when compared to non-sterile compositions. This provides a valuable safety measure, especially when subjects receiving the compositions have immune defects, are suffering from infection, and/or are susceptible to infection.

### Administration

Administration according to the present invention may be by a variety of routes, including but not limited to subcutaneous, intravenous, and intraperitoneal routes. For example, the mode of administration for the composition of any one of the treatment methods provided may be by intravenous administration, such as an intravenous infusion that, for example, may take place over about 1 hour. The compositions referred to herein may be manufactured and prepared for administration using conventional methods.

The compositions of the invention can be administered in effective amounts, such as the effective amounts described herein. In some embodiments of any one of the methods or compositions provided, repeated multiple cycles of administration of synthetic nanocarriers comprising an immunosuppressant is undertaken. Doses of dosage forms may contain varying amounts of immunosuppressants according to the invention. The amount of immunosuppressants present in the dosage forms can be varied according to the nature of the synthetic nanocarrier and/or immunosuppressant, the therapeutic benefit to be accomplished, and other such parameters. In embodiments, dose ranging studies can be conducted to establish optimal therapeutic amounts of the component(s) to be present in dosage forms. In embodiments, the component(s) are present in dosage forms in an amount effective to induce or increase autophagy or generate a preventative or therapeutic response to an autophagy-associated disease or disorder. Dosage forms may be administered at a variety of frequencies.

Aspects of the invention relate to determining a protocol for the methods of administration as provided herein. A protocol can be determined by varying at least the frequency, dosage amount of the synthetic nanocarriers comprising an immunosuppressant and subsequently assessing a desired or undesired therapeutic response, such as the induction and/or increase in autophagy. The protocol can comprise at least the frequency of the administration and doses of the synthetic nanocarriers comprising an immunosuppressant. Any one of the methods provided herein can include a step of determining a protocol or the administering steps are performed according to a protocol that was determined to achieve any one or more of the desired results as provided herein.

The compositions provided herein, comprising synthetic nanocarriers comprising an immunosuppressant, in some embodiments, are not administered concomitantly (e.g., simultaneously) with a therapeutic macromolecule, viral vector, or APC presentable antigen or are administered concomitantly with a combination of a therapeutic macromolecule, viral vector, or APC presentable antigen and a separate administration (e.g., not in the same administered composition and/or administered separately for a different purpose such as not for inducing or increasing autophagy) of synthetic nanocarriers comprising an immunosuppressant. In some embodiments, the compositions provided herein, comprising synthetic nanocarriers coupled to an immunosuppressant, are not administered within 1 month, 1 week, 6 days, 5, days, 4 days, 3 days, 2 days, 1 day, 12 hour, 6 hours, 5 hours, 4 hours, 3 hours, 2 hours, or 1 hour of a therapeutic macromolecule, viral vector, or APC presentable antigen. In some embodiments of the foregoing, when administered concomitantly with another therapeutic, the synthetic nanocarriers comprising an immunosuppressant are for an effect provided herein and not for a different purpose (or at least not solely) and/or not for an effect on the other therapeutic (or at least not solely). In some embodiments, when the other therapeutic and the synthetic nanocarriers comprising an immunosuppressant are not administered concomitantly, the synthetic nanocarriers comprising an immunosuppressant do not have an effect or a clinically meaningful or substantial effect on the other therapeutic, such as that is achieved when the nanocarriers comprising an immunosuppressant are administered concomitantly with the other therapeutic. In some embodiments, when the other therapeutic and the synthetic nanocarriers comprising an immunosuppressant are both administered concomitantly or not, the synthetic nanocarriers comprising an immunosuppressant have a clinically significant effect on autophagy alone or in addition to another effect, such as on the other therapeutic.

In some embodiments, when the other therapeutic and the synthetic nanocarriers comprising an immunosuppressant are not administered concomitantly or concomitantly but for a purpose provided herein, the effect of the synthetic nanocarriers comprising an immunosuppressant on the other therapeutic is not needed or is an additional effect (when administered concomitantly). In some embodiments, when the other therapeutic and the synthetic nanocarriers comprising an immunosuppressant are not administered concomitantly, the synthetic nanocarriers comprising an immunosuppressant do not have an effect or a clinically meaningful or substantial effect on the other therapeutic that is achieved when the nanocarriers comprising an immunosuppressant are administered concomitantly with the other therapeutic (e.g., increased efficacy of the other therapeutic).

The compositions and methods described herein can be used for subjects having or at risk of having one or more autophagy-associated diseases or disorders. Examples of autophagy-associated diseases and disorders include, but are not limited to, metabolic syndrome, liver disease, and inborn errors of metabolism (organic acidemias, methylmalonic acidemia, propionate acidemia, ornithine transcarbamylase deficiency).

Examples of liver diseases include, but are not limited to metabolic liver disease (e.g., nonalcoholic fatty liver disease (NAFLD) and nonalcoholic steatohepatitis (NASH)); alcohol-related liver disease (e.g., fatty liver, alcoholic hepatitis); autoimmune liver diseases (e.g., autoimmune hepatitis, primary biliary cirrhosis, primary sclerosing cholangitis); a viral infection (e.g., hepatitis A, B, or C); liver cancer (e.g., hepatocellular carcinoma, HCC); an inherited metabolic disorder (e.g., Alagille syndrome, alpha-1 antitrypsin deficiency, Crigler-Najjar syndrome, galactosemia, Gaucher disease, a urea cycle disorder (e.g., ornithine transcarbamylase (OTC) deficiency), Gilbert syndrome, hemochromatosis, Lysosomal acid lipase deficiency (LAL-D), organic acidemia (e.g., methylmalonic acidemia), Reye syndrome, Type I Glycogen Storage Disease, and Wilson's disease); drug hepatotoxicity (e.g., from exposure to acetaminophen, non-steroidal anti-inflammatory drugs (NSAIDs, aspirin, ibuprofen, naproxen sodium, statins, antibiotics, e.g., amoxicillin-clavulanate or erythromycin, arthritis drugs, e.g., methotrexate or azathioprine, antifungal drugs, niacin, steroids, allopurinol, antiviral drugs, chemotherapy, herbal supplements, e.g., aloe vera, black cohosh, cascara, chaparral, comfrey, ephedra, or kava, vinyl chloride, carbon tetrachloride, paraquat, or polychlorinated biphenyls); and fibrosis (e.g., cirrhosis).

Inborn errors of metabolism include, but are not limited to organic acidemias, methylmalonic acidemia, propionate acidemia, urea cycle disorders, ornithine transcarbamylase deficiency, citrillinemia, homocystinuria, galactosemia, maple sugar urine disease (MSUD), phenylketonuria, glycogen storage disease types 1-13, G6PD deficiency, glutaric acidemia, tyrosinemia, disorders of amino acid metabolism, disorders of lipid metabolism, disorders of carbohydrate metabolism.

### Dosing

The compositions provided herein may be administered according to a dosing schedule. Provided herein are a number of possible dosing schedules. Accordingly, any one of the subjects provided herein may be treated according to any one of the dosing schedules provided herein. As an example, any one of the subject provided herein may be treated with a composition comprising synthetic nanocarriers comprising an immunosuppressant, such as rapamycin, according to any one of these dosage schedules.

### EXAMPLES

### Example 1: Synthesis of Synthetic Nanocarriers Comprising an Immunosuppressant (Prophetic)

Synthetic nanocarriers comprising an immunosuppressant, such as rapamycin, can be produced using any method known to those of ordinary skill in the art. Preferably, in some embodiments of any one of the methods or compositions provided herein the synthetic nanocarriers comprising an immunosuppressant are produced by any one of the methods of US Publication No. US 2016/0128986 A1 and US Publication No. US 2016/0128987 A1, the described methods of such production and the resulting synthetic nanocarriers being incorporated herein by reference in their entirety. In any one of the methods or compositions provided herein, the synthetic nanocarriers comprising an immunosuppressant are such incorporated synthetic nanocarriers.

### Example 2: Administration of Synthetic Nanocarriers Coupled to Immunosuppressant Prior to or after Treatment with Inflammatory Agent

There are several accepted models of studying liver failure induced by drug toxicity and inflammatory reactions of chronic and acute nature in laboratory models, one of which involves challenging mice with sublethal amounts of polyclonal T cell activator, concanavalin A (Con A), which induces profound liver injury and has been often used for the study of pathophysiology of liver damage in human liver diseases, specifically autoimmune and viral hepatitis (Tiegs et al., 1992; Miyazava et al., 1998). Mice treated with Con A immediately manifest key clinical and biochemical features of liver failure characterized by a marked increase in the levels of transaminases in serum and massive infiltration of lymphocytes into the liver leading to death of extensive hepatocyte necrosis (Zhang et al., 2009). While pre-treatment with systemic doses of a variety of immunosuppressive compounds have been shown to be beneficial against a Con A challenge, these interventions are neither liver-specific nor practical.

Three groups of wild-type BALB/c female mice were injected intravenously (i.v.) with Con A (12 mg/g) either alone or with an intravenous injection of synthetic nanocarriers coupled to immunosuppressant like those of Example 1, e.g., ImmTOR™, at 200 μg of rapamycin one hour prior to or one hour following the Con A injection. Twenty-four hours later, the animals were terminally bled and the serum concentration of alanine aminotransferase (ALT) was measured using a mouse alanine aminotransferase activity colorimetric/fluorometric assay (Biovision, Milpitas, Calif.).

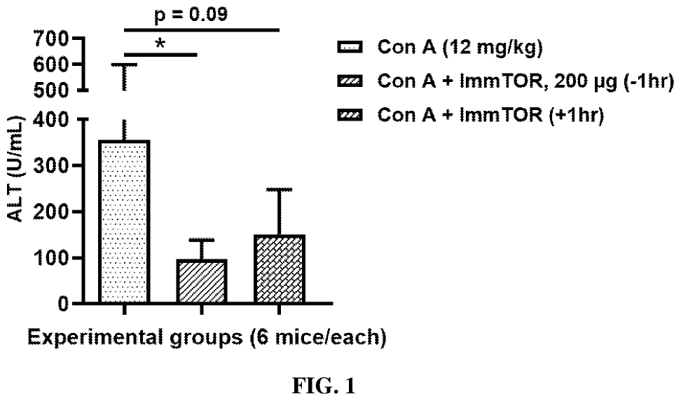

While nearly all the mice that only received an injection of Con A showed a profound ALT elevation, the ALT level was much lower in mice treated with ImmTOR™ whether preventively (one hour before the Con A challenge) or therapeutically (one hour after the Con A challenge) (FIG. 1). This demonstrates that a single intravenous injection of ImmTOR™ either before or after Con A administration provides a significant benefit against Con A-induced toxicity.

### Example 3: Synthetic Nanocarriers Coupled to Immunosuppressant Reduce Urinary Orotic Acid Levels in a Mouse Model of Ornithine Transcarbamylase (OTC) Deficiency

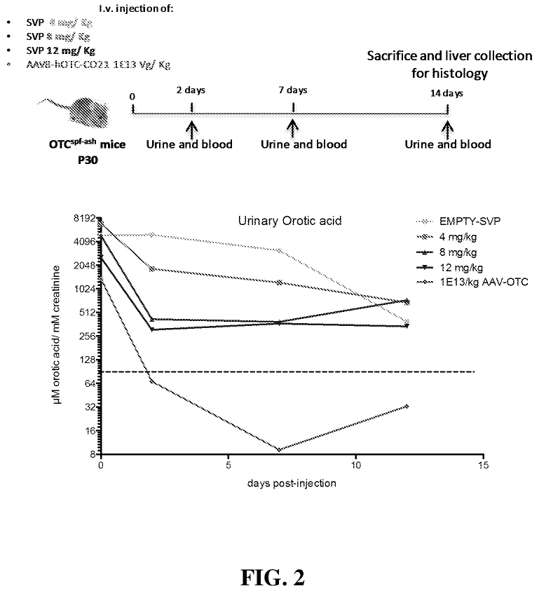

OTCspf^{ash} mice, a mouse model for OTC deficiency, were treated with a single injection of synthetic nanocarriers like those of Example 1, e.g., ImmTOR™, at doses of 4, 8, or 12 mg/kg or with empty nanocarriers 30 days after birth (FIG. 2). A positive control group of mice received a high dose of AAV8 gene therapy vector expressing the OTC gene under control of a liver-specific promoter. OTCspf^{ash} mice treated with ImmTOR™ showed a rapid and dose-dependent decline of urinary orotic acid within 2 days after dosing. The decline in urinary orotic acid was substantial, although the decline was not as low as that observed after AAV-OTC gene therapy (FIG. 2).

### Example 4: ImmTOR™ Application Prior to or after Treatment with Hepatotoxic Agent Acetaminophen (APAP) Leads to a Decrease of Serum Concentration of Alanine Transferase in Wild-Type Mice

Liver failure induced by drug toxicity is a major medical and social issue. One of its main causes is overdosing with acetaminophen (APAP), which is one of the most frequently used drugs and an overdose of which may lead to hepatotoxicity and acute liver failure (ALF). More specifically, APAP-induced hepatotoxicity remains the most common cause of ALF in many countries including the US (Lee W N; Clin. Liver Dis. 2013, 17:575-586). At the same time, APAP-induced acute hepatic damage is one of the most commonly used experimental models of acute liver injury in mice known to result in a highly reproducible, dose-dependent hepatotoxicity. Moreover, this model possesses strong translational value since the outcomes of mouse APAP-induced liver injury (AILI) studies are directly transferable to humans (Mossanen and Tacke, Lab. Animals, 2015, 49:30-36).

The main cause of AILI is the massive necrosis of hepatocytes. In humans, APAP is metabolized in the liver, which may lead to creation of a toxic N-acetyl-p-benzoquinone imine (NAPQI), which is normally converted by the antioxidant glutathione (GSH) into a harmless reduced form. However, when the amount of metabolized APAP increases due to an overdose and GSH is depleted, then elevated NAPQI binds to mitochondrial proteins forming cytotoxic protein adducts, leading to hepatocyte necrosis. This in turn may be followed by sterile inflammation as a response to hepatocyte necrosis, which leads to the massive release of danger-associated molecular patterns and the inflammasome formation in many innate immune cells. Such activation of innate immune system results in the recruitment of immune cells to inflammation site and further enhances hepatocyte necrosis. All of these stages, including NAPQI accumulation, hepatocyte necrosis, and strong inflammatory response, are well recapitulated in the AILI model in mice (Mossanen ans Tacke, 2015).

Since APAP-induced oxidative stress and mitochondrial dysfunction plays a central role in the pathogenesis of AILI, the US FDA recommends N-acetyl cysteine, an antioxidant, as the only therapeutic option for APAP-overdosed patients; however, this medication has limitations including adverse effects and narrow therapeutic window and if it is missed, liver transplantation is the only choice to improve survival in AILI patients (Yan et al., Redox Biology, 2018, 17:274-283). Therefore, the development of new drugs against AILI is clearly needed. Here we show that a single intravenous injection of synthetic nanocarriers like those of Example 1, e.g., ImmTOR™, either before or after APAP administration provides a significant benefit against AILI in wild-type mice.

Three groups of wild-type BALB/c female mice were injected (i.v.) with APAP (350 mg/kg) either alone or with ImmTOR™ at 200 μg of rapamycin injected (i.v.) either at 1 hr prior to or 1 hr after APAP injection. 24 hours later animals were terminally bled and serum concentration of alanine aminotransferase (ALT) measured using mouse alanine aminotransferase activity colorimetric/fluorometric assay (Biovision, Milpitas, Calif.). While nearly all mice not treated with ImmTOR™ showed a profound ALT elevation, ALT level was much lower in mice treated with ImmTOR™ whether preventively, or, importantly, therapeutically, i.e. after APAP challenge (FIG. 3).

### Example 5: Synthetic Nanocarriers Coupled to Immunosuppressant Reduce Urinary Orotic Acid Levels in a Mouse Model of Ornithine Transcarbamylase (OTC) Deficiency

Neutralizing antibodies (NAbs) are formed in response to AAV vector administration, preventing the ability to repeat vector administration in pediatric patients who need one or more additional doses to achieve or sustain efficacy. As a result, the tolerability and efficacy of synthetic nanocarriers like those of Example 1, e.g., ImmTOR™, in juvenile OTC^{spf-ash} mice was evaluated.

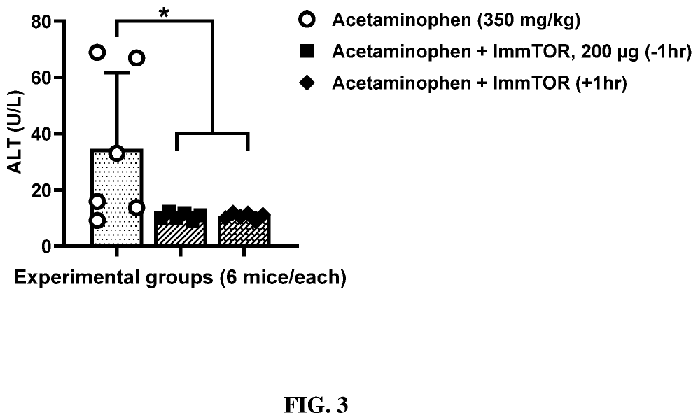

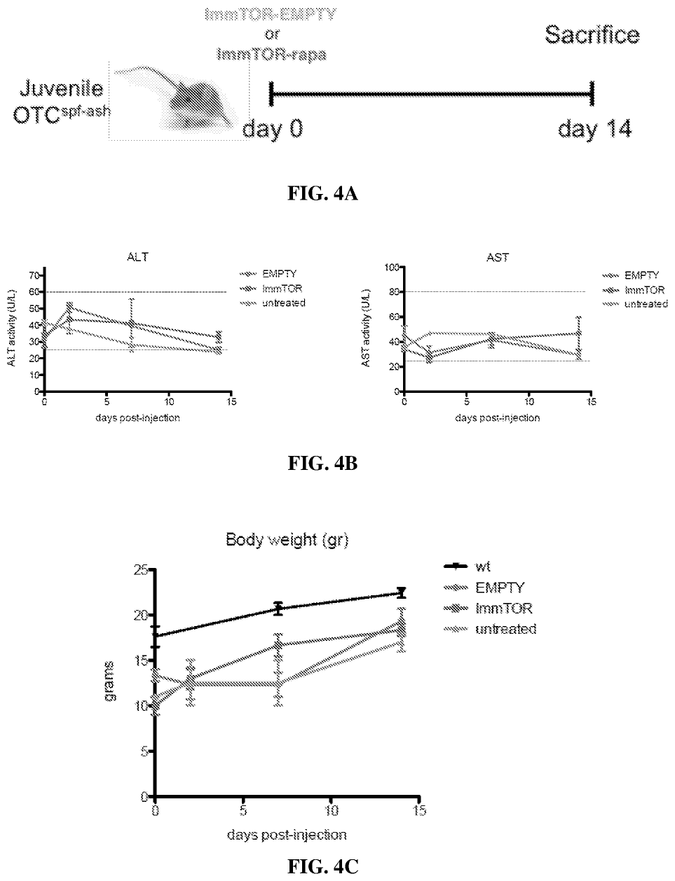

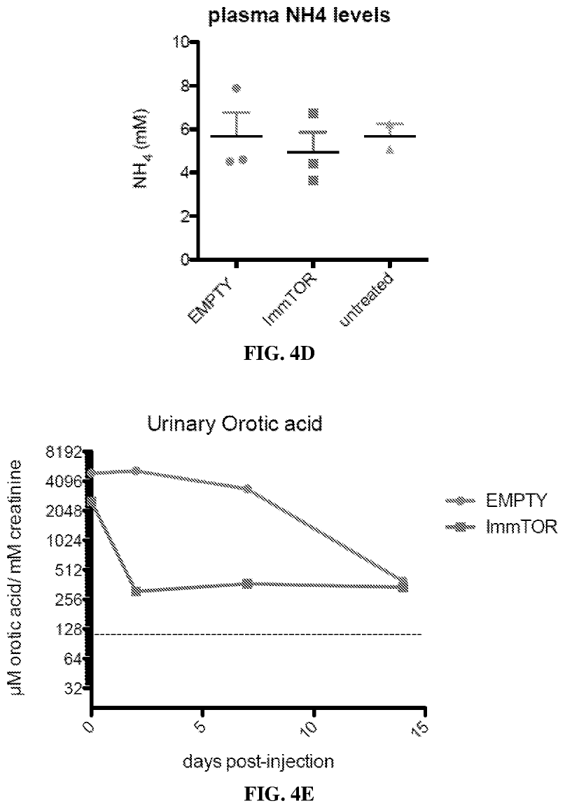

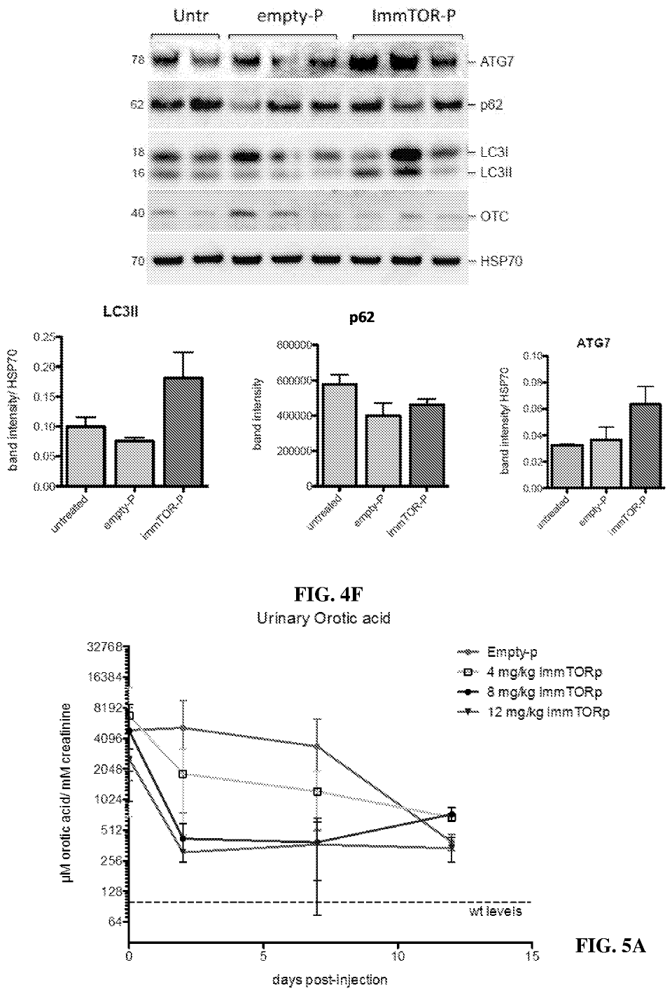

A tolerability study of ImmTOR™ in juvenile OTC^{spf-ash} mice was performed. EMPTY-nanocarriers or ImmTOR™ were i.v. injected in OTC^{spf-ash} juvenile mice (FIG. 4A). After 14 days, injected mice were tested for: ALT and AST (FIG. 4B) body weight (FIG. 4C), plasma ammonia levels (FIG. 4D), Urinary Orotic acid (FIG. 4E) and autophagy markers in liver lysates of treated mice (FIG. 4F), all demonstrating that ImmTOR™ alone have a benefit in the OTC^{spf-ash} model as indicated by OTC decrease and autophagy induction without any noticeable side-effects.

Notably, a single dose of ImmTOR™ administered to OTC^{spf-ash} mice induced autophagy biomarkers hepatic LC3II and ATG7 and reduced autophagy biomarker p62, consistent with an increase in autophagy. This demonstrates that, in a mouse model of OTC deficiency, a single injection of ImmTOR™ decreases urinary orotic acid and that this decrease is associated with an increase in autophagy.

### Example 6: Tolerability Study of Synthetic Nanocarriers Coupled to Immunosuppressant in Mouse Model of Ornithine Transcarbamylase (OTC) Deficiency

To evaluate the safety of synthetic nanocarriers like those of Example 1, e.g., ImmTOR™, in the mouse model for OTC deficiency OTC^{Spf-Ash}, juvenile OTC^{Spf-Ash} mice (30 days old) were intravenously (IV) injected with ImmTOR™. Five experimental groups were tested: administration of 4 mg/kg ImmTOR™, administration of 8 mg/kg ImmTOR™, administration of 12 mg/kg ImmTOR™, administration of empty nanocarriers, and untreated animals.

The mice were weighed daily, and samples of urine and blood were collected 2, 7, and 14 days after the injection. The mice were sacrificed 14 days after the injection. Aspartate aminotransferase (AST) and alanine aminotransferase (ALT) activity were measured in plasma using a Sigma kit (MAK055 and MAK052), and urinary orotic acid was measured by HPLC-MS.

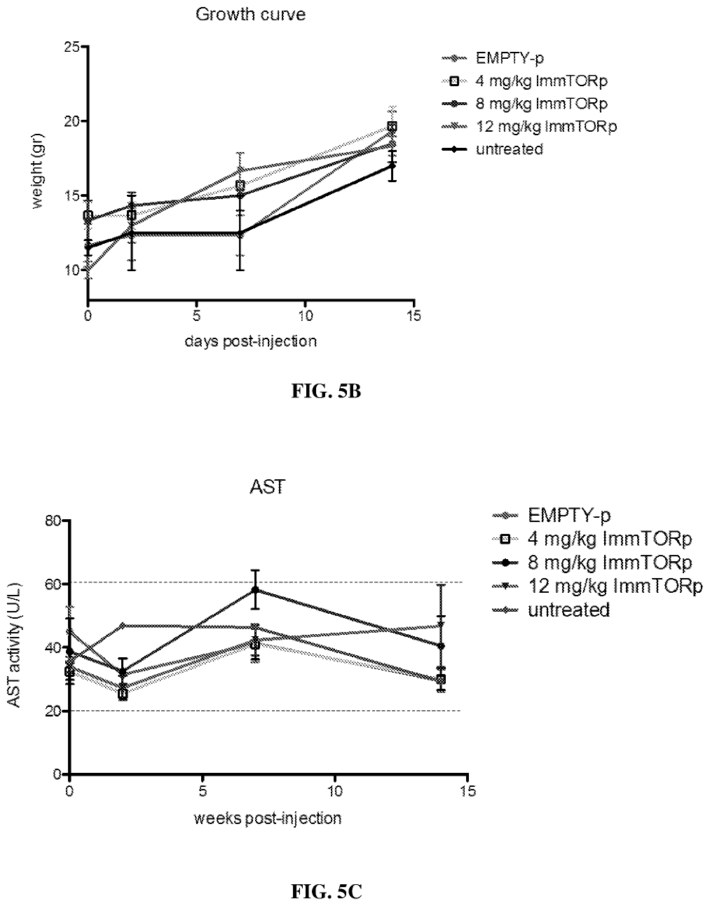

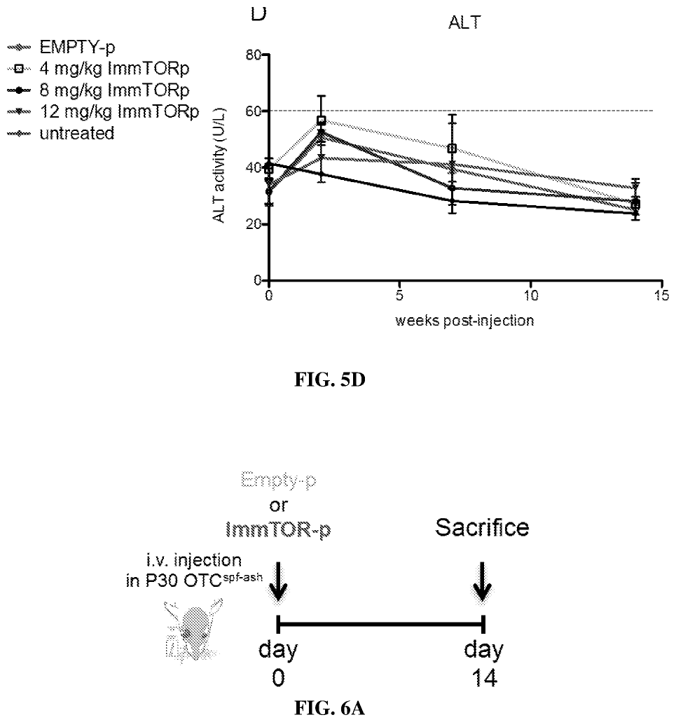

Transaminase (e.g., AST and ALT) values remained within the physiological range after ImmTOR™ administration, indicating that treatment is well-tolerated in young OTC^{Spf-Ash} mice (FIGS. 5C-5D). Moreover, a dose-dependent improvement of the urinary orotic acid, an OTC deficiency marker, was observed. The groups injected with 8 mg/kg and 12 mg/kg ImmTOR™ doses showed a reduction in urinary orotic acid compared to mice treated with empty nanocarriers (FIG. 5A). At the latest time point (14 days post injection), the effect was lost and all groups presented similar urinary orotic acid levels.

In all, these data illustrate that ImmTOR™ can be safely administered to juvenile OTC^{Spf-Ash} mice.

### Example 7: Synthetic Nanocarriers Reduce Urinary Orotic Acid and Hepatic Ammonia in OTC^{spf-ash} Mice Via Autophagy Activation

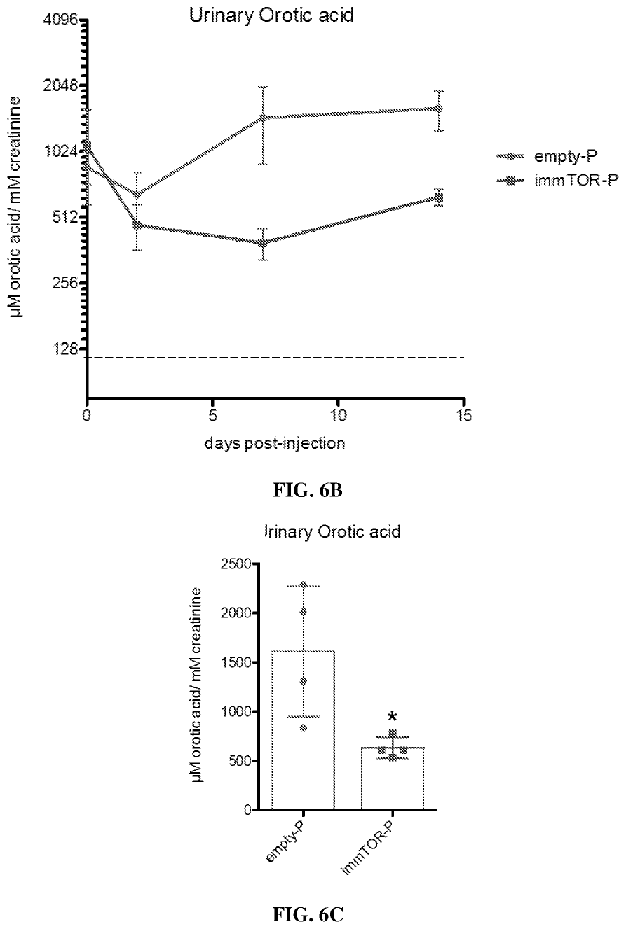

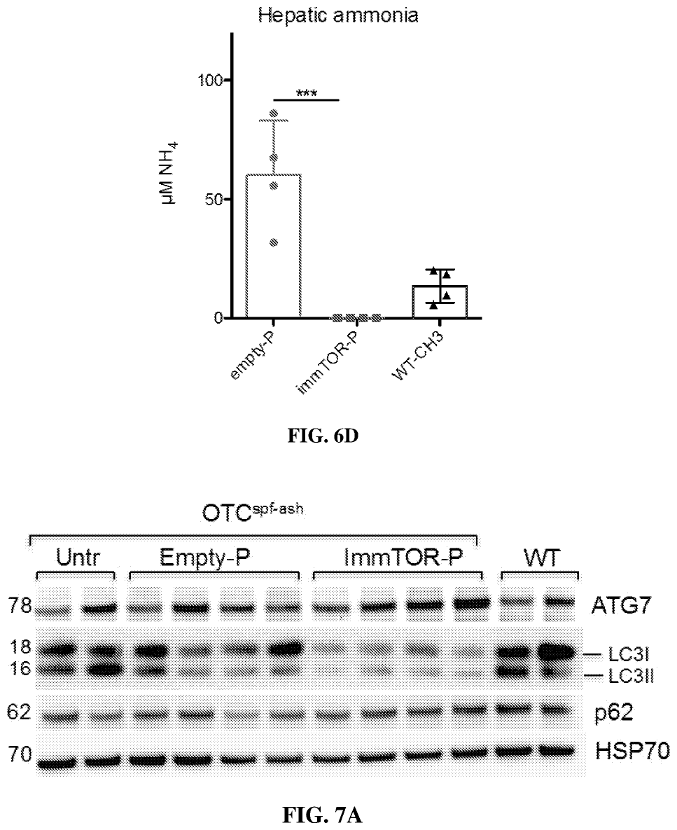

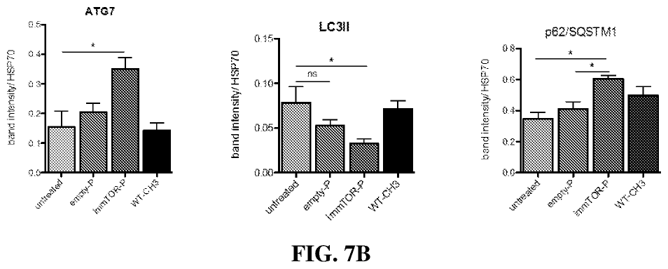

To further investigate and confirm the beneficial effect of synthetic nanocarriers like those of Example 1, e.g., ImmTOR™, in the OTC^{Spf-Ash} phenotype, juvenile OTC^{Spf-Ash} mice (30 days old) were intravenously (IV) with 12 mg/kg ImmTOR™ or 12 mg/kg of empty nanocarriers (FIG. 6A). Injections were performed retro-orbitally. Urine samples were collected 2, 7, and 14 days post-injection. Mice were sacrificed at 14 days post-injection and livers were collected. Analysis of urinary orotic acid showed a two-fold reduction of urinary orotic acid in the ImmTOR™-treated animals (FIG. 6B), which was maintained for 14 days (FIG. 6C). At sacrifice, the liver was collected and pulverized. Total lysates were prepared. The liver lysates were quantified by Bradford assay and an equal amount of lysate was used to quantify ammonia using an ammonia assay kit (Sigma AA0100). ImmTOR™-treated animals showed a reduction of ammonia in the liver 50 times that of the empty nanocarrier-treated animals (FIG. 6D).

The data demonstrate that a dose of 12 mg/kg of ImmTOR™ was able to statistically reduce the main markers of OTC deficiency (orotic acid and ammonia) in the OTC^{Spf-Ash} model. In particular, orotic acid was reduced 2-fold in urine, and the liver was completely detoxified from ammonia.

To investigate the possibility that ImmTOR™ were reducing urinary orotic acid and ammonia levels via autophagy activation in the liver, autophagy markers in the liver of ImmTOR™ or empty nanocarrier-treated mice were analyzed.

Livers from ImmTOR™-treated and empty nanocarrier-treated animals were pulverized with a mortar, and total liver protein lysates were prepared from the powder with a lysis buffer containing 0.5% Triton-x, 10 mM Hepes pH 7.4, and 2 mM dithiothreitol. Ten (10) μg of liver lysate were analyzed by Western blot with antibodies recognizing LC3II, ATG7 and p62, the most common markers of autophagy (FIG. 6A).

Notably, livers harvested from ImmTOR™-treated animals showed an increase in the ATG7 autophagy marker and a decrease in LC3II and p62 markers (FIG. 6B), indicating an activation of the autophagy flux after ImmTOR™ administration.

These data support that ImmTOR™ activate the hepatic autophagy flux in OTC^{Spf-Ash} mice, contributing to the reduction in OTC deficiency clinical manifestations.

What is claimed is:

1. A method of inducing or increasing autophagy in a subject and/or treating or preventing an autophagy-associated disease or disorder in the subject comprising:
administering a composition comprising synthetic nanocarriers comprising an immunosuppressant to the subject;
wherein the subject has a need for the induction or increase in autophagy and/or has or is at risk of developing the autophagy-associated disease or disorder.

2. The method of claim 1, wherein the administration of the synthetic nanocarriers comprising the immunosuppressant increases autophagy in the liver.

3. The method of claim 1 or claim 2, wherein the synthetic nanocarriers comprising the immunosuppressant are not administered concomitantly with a therapeutic macromolecule.

4. The method of claim 3, wherein the synthetic nanocarriers comprising the immunosuppressant are not administered simultaneously with the therapeutic macromolecule.

5. The method of any one of claims 1-4, wherein the synthetic nanocarriers comprising the immunosuppressant are not administered concomitantly with a viral vector.

6. The method of claim 8, wherein the synthetic nanocarriers comprising the immunosuppressant are not administered simultaneously with the viral vector.

7. The method of any one of claims 1-6, further comprising administering a viral vector, therapeutic macromolecule or APC presentable antigen.

8. The method of any one of claims 1-7, wherein the synthetic nanocarriers comprising the immunosuppressant are not administered concomitantly with an APC presentable antigen.

9. The method of claim 8, wherein the synthetic nanocarriers comprising the immunosuppressant are not administered simultaneously with the APC presentable antigen.

10. The method of claim 1 or claim 2, wherein the synthetic nanocarriers comprising the immunosuppressant are not administered concomitantly with another therapeutic to treat or prevent the autophagy-associated disease or disorder.

11. The method of claim 1 or claim 2, wherein the synthetic nanocarriers comprising the immunosuppressant are not administered simultaneously with another therapeutic to treat or prevent the autophagy-associated disease or disorder.

12. The method of any one of the preceding claims, wherein the method further comprises identifying and/or providing the subject having or suspected of having the autophagy-associated disease or disorder.

13. The method of any one of the preceding claims, wherein the autophagy-associated disease or disorder is a liver diseases.

14. The method of any one of the preceding claims, wherein the immunosuppressant is an mTOR inhibitor.

15. The method of claim 14, wherein the mTOR inhibitor is rapamycin or a rapalog.

16. The method of any one of the preceding claims, wherein the immunosuppressant is encapsulated in the synthetic nanocarriers.

17. The method of any one of the preceding claims, wherein the synthetic nanocarriers comprise lipid nanoparticles, polymeric nanoparticles, metallic nanoparticles, surfactant-based emulsions, dendrimers, buckyballs, nanowires, virus-like particles or peptide or protein particles.

18. The method of claim 17, wherein the synthetic nanocarriers comprise polymeric nanoparticles.

19. The method of claim 18, wherein the polymeric nanoparticles comprise a polyester, polyester attached to a polyether, polyamino acid, polycarbonate, polyacetal, polyketal, polysaccharide, polyethyloxazoline or polyethyleneimine.

20. The method of claim 19, wherein the polymeric nanoparticles comprise a polyester or a polyester attached to a polyether.

21. The method of claim 19 or 20, wherein the polyester comprises a poly(lactic acid), poly(glycolic acid), poly(lactic-co-glycolic acid) or polycaprolactone.

22. The method of any one of claims 19-21, wherein the polymeric nanoparticles comprise a polyester and a polyester attached to a polyether.

23. The method of any one of claims 19-22, wherein the polyether comprises polyethylene glycol or polypropylene glycol.

24. The method of any one of the preceding claims, wherein the mean of a particle size distribution obtained using dynamic light scattering of a population of the synthetic nanocarriers is a diameter greater than 110 nm.

25. The method of claim 24, wherein the diameter is greater than 150 nm.

26. The method of claim 25, wherein the diameter is greater than 200 nm.

27. The method of claim 26, wherein the diameter is greater than 250 nm.

28. The method of any one of claims 24-27, wherein the diameter is less than 5 μm.

29. The method of claim 28, wherein the diameter is less than 4 μm.

30. The method of claim 29, wherein the diameter is less than 3 μm.

31. The method of claim 30, wherein the diameter is less than 2 μm.

32. The method of claim 31, wherein the diameter is less than 1 μm.

33. The method of claim 32, wherein the diameter is less than 750 nm.

34. The method of claim 33, wherein the diameter is less than 500 nm.

35. The method of claim 34, wherein the diameter is less than 450 nm.

36. The method of claim 35, wherein the diameter is less than 400 nm.

37. The method of claim 36, wherein the diameter is less than 350 nm.

38. The method of claim 37, wherein the diameter is less than 300 nm.

39. The method of any one of the preceding claims, wherein the load of immunosuppressant comprised in the synthetic nanocarriers, on average across the synthetic nanocarriers, is between 0.1% and 50% (weight/weight).

40. The method of claim 39, wherein the load is between 4% and 40%.

41. The method of claim 40, wherein the load is between 5% and 30%.

42. The method of claim 41, wherein the load is between 8% and 25%.

43. The method of any one of the preceding claims, wherein an aspect ratio of a population of the synthetic nanocarriers is greater than or equal to 1:1, 1:1.2, 1:1.5, 1:2, 1:3, 1:5, 1:7 or 1:10.
